(19) United States
(12) Patent Application Publication
(10) Pub. No.: US 2025/0269366 A1
(43) Pub. Date: Aug. 28, 2025

(54) CONTINUOUS ION EXCHANGE AND ESTERIFICATION OF FERMENTED MALONIC ACID

(71) Applicant: LYGOS, INC., BERKELEY, CA (US)

(72) Inventors: Nicholas Ohler, Berkeley, CA (US); Johan Van Walsem, Berkeley, CA (US); Chi Le, Berkeley, CA (US); Kelvin Shing, Berkeley, CA (US); Daniel Blackburn, Berkeley, CA (US); Owen Budavich, Berkeley, CA (US)

(73) Assignee: LYGOS, INC., BERKELEY, CA (US)

(21) Appl. No.: 18/027,336

(22) PCT Filed: Sep. 20, 2021
(86) PCT No.: PCT/US2021/051094

§ 371 Date: Mar. 20, 2023

Related U.S. Application Data
(60) Provisional application No. 63/081,211, filed on Sep. 21, 2020.

(51) Int. Cl. B01J 49/60; B01J 39/07; B01J 41/05; B01J 49/07; B01J 49/53; C07C 29/78; C07C 29/80

(52) U.S. Cl. CPC B01J 49/60; B01J 39/07; B01J 41/05; B01J 49/07; B01J 49/53; C07C 29/78; C07C 29/80

(57) ABSTRACT

Provided herein is a process of ion exchange comprising malonic acid or a salt thereof and a cation or an anion cation exchange resin. The ion exchange is accomplished, e.g., and without limitation by continuous ion exchange. A valve and resin bed configuration is useful in this regard. The malonic acid separated by ion exchange is esterified, e.g., by Fisher esterification by using an acid and an alcohol.

### CROSS-REFERENCE TO RELATED APPLICATIONS

This application is a § 371 National Stage Application of International Application No. PCT/US2021/051094, filed Sep. 20, 2021, which claims priority to and the benefit of U.S. Provisional Application No. 63/081,211, filed Sep. 21, 2020, each of which are herein incorporated by reference in their entirety.

### FIELD OF THE DISCLOSURE

Provided herein is a process of ion exchange comprising malonic acid or a salt thereof and a cation or an anion cation exchange resin. The ion exchange is accomplished, e.g., and without limitation by continuous ion exchange. A valve and resin bed configuration is useful in this regard. The malonic acid separated by ion exchange is esterified, e.g., by Fisher esterification by using an acid and an alcohol.

### SUMMARY OF THE DISCLOSURE

Provided herein is a process of ion exchange comprising malonic acid or a salt thereof and a cation or an anion cation exchange resin. The cation exchange resin is regenerated periodically with an acid to convert the resin to the protonated form ready to absorb the cation of the malonate salt (e.g. sodium, ammonium, and the like) so that the malonic acid salt is converted into the corresponding acid in the raffinate leaving the ion exchange. Upon regeneration of the cation resin with acid, typically a strong mineral acid is used, the cation is eluted as the corresponding salt of the acid with the resin being regenerated to the proton form. The anion exchange resin is regenerated periodically with an acid, typically a mineral acid that is a stronger acid relative to malonic acid based on having a lower pKa, so that the malonic acid salt is protonated and an aqueous malonic acid solution is eluted from the resin by the acid regeneration. The ion exchange is accomplished, e.g., and without limitation by continuous ion exchange. A valve and resin bed configuration is useful in this regard. Whether cation or anion exchange is used the incoming malonate salt is converted into a malonic acid stream without a salt form thereof, i.e., without a counterion (cation) and a separate stream containing the corresponding cation salt of the regenerating acid. The malonic acid separated by ion exchange is esterified, e.g., by Fisher esterification by using an acid and an alcohol. Suitable alcohols include lower alkanols, such as C₁-C₉ alkanols, e.g. C₁-C₆ alkanols, and aromatic alcohols such as phenols. Preferred alcohols include methyl alcohol and ethyl alcohol.

In one aspect, provided herein is a process of ion exchange comprising: contacting malonic acid or a salt thereof and a cation or an anion cation exchange resin, wherein the cation exchange resin is regenerated periodically with an acid into the protonated form so that the malonic acid salt is protonated while contacting the resin and is included in a raffinate stream as an aqueous malonic acid solution free of a cation and the cation is adsorbed onto the resin to be eluted as the corresponding salt of the regenerating acid, or wherein the anion exchange resin is regenerated periodically with an acid so that the malonic acid that has previously been adsorbed on the resin is eluted from the resin and the regenerating acid is adsorbed on the resin ready for the next cycle, wherein the ion exchange is accomplished by continuous ion exchange.

In one embodiment, the malonic acid salt comprises a sodium, calcium, or ammonium salt.

In another embodiment, the malonic acid or salt thereof is included in a crude aqueous malonate fermentation product. As used herein, a crude aqueous malonate fermentation product refers to an aqueous mixture containing malonic acid or a salt thereof, which is obtained by a fermenting a feedstock such as, without limitation, glucose, by a microorganism, such as a unicellular organism, such as yeast. Pichia kudriavzevii is a preferred microorganism.

In another embodiment, the malonic acid or salt thereof is included in a crude aqueous malonate fermentation product, which is separated from fermenting cells by microfiltration, centrifugation, drum filtration, or belt filtration. In another embodiment, the malonic acid or salt thereof is included in a crude aqueous malonate fermentation product, which is separated from fermenting by filtering a crude aqueous malonate fermentation product through an ultrafilter or nanofilter. In another embodiment, a nanofilter is utilized and the nanofilter material is selected so that it rejects >50% of trehalose contained in the fermentation broth. In another embodiment, a nanofilter is utilized and the nanofilter material is selected so that it rejects >10% or >30% of the glucose contained in the fermentation broth. In another embodiment, a nanofilter is utilized and the nanofilter material is selected so that it rejects >10% or >30% of the succinate salts contained in the fermentation broth.

In another embodiment, the continuous ion exchange is accomplished using a valve and resin bed configuration designed to simulate a moving resin bed.

In another embodiment, the ion exchange is performed with a cation exchange resin that is regenerated periodically with acid so that the malonate is protonated while contacting the resin during the cation adsorption stage. In another embodiment, the fermentation product contains malonate primarily as an ammonium salt, so that ammonium sulfate is generated as a co-product when the resin is periodically regenerated. In another embodiment, the ion exchange is performed with an anion exchange resin that is regenerated periodically with acid so that the malonate is protonated and eluted from the resin by the acid regeneration. In another embodiment, the acid used for regeneration is aqueous sulfuric acid. In another embodiment, impurities of high molecular weight pass substantially through the anion exchange resin during malonate adsorption on the anion exchange resin, thereby separating the impurities from malonate. In another embodiment, a fermenting cell such as yeast and impurities of high molecular weight pass substantially through the anion exchange resin during malonate adsorption on the anion exchange resin, thereby separating the fermenting cells and impurities from the malonate. In another embodiment, the malonate salt comprises an ammonium salt. In some embodiments, ammonium sulfate is generated and eluted as a co-product while malonate is adsorbed onto the resin.

In some embodiments, for separating malonic acid from an aqueous solution containing ammonium malonate, a continuous process using cationic ion exchange resin is preferred over the batch process involving a conventional column chromatography using, e.g., cationic ion exchange resin. Without being bound by theory, in the continuous ion exchange, the adsorption and desorption operations are continuously occurring. Accordingly, there is a continuous use of feed accompanied by a continuous production of extract such as malonic acid. As will be clear to those skilled in the art, such continuous operation results in lower resin inventory, better utilization of chemicals with near stoichiometric operation possible, less dilution due to washing operations and ability to maximize loading of the resin without losing any valuable product due to breakthrough from the column. In one embodiment, the continuous ion exchange chromatography is operated either as a moving port system or as a moving column system. The moving port system includes a vertical column Subdivided into a number of interlinked compartments and the fluid inlets and outlets of each compartment are controlled by specifically designed rotary master valve. A moving column system includes multiple chromatography columns mounted on a rotary carousel.

### BRIEF DESCRIPTION OF THE FIGURES

FIG. 1 illustrates a breakthrough curve of malonate and sulfate using Amberlyst A-26 strong base resin;

FIG. 2 illustrates a breakthrough curve of malonate and sulfate using Amberlyst A-22 weak base resin;

FIG. 3 illustrates a breakthrough curve of sulfate using Purolite-C160H strong acid resin;

FIG. 4 illustrates a conversion of Malonic acid versus residence time in the fixed-bed reactor; and

FIG. 5 illustrates a rate of hydrolysis of dimenthyl malonate versus temperature under methanol/water distillation conditions.

### DETAILED DESCRIPTION OF THE FIGURES

### Definition

Throughout this disclosure, various publications, patents, published patent applications, and the likes may be referenced by an identifying citation. The disclosures of these publications, patents and published patent applications are hereby incorporated by reference into the present disclosure in their entirety to more fully describe the state of the art to which this invention pertains.

The practice of the present technology will employ, unless otherwise indicated, certain conventional techniques of organic chemistry and chemical engineering which are within the skill of an artisan.

As used in the specification and claims, the singular form “a,” “an” and “the” include plural references unless the context clearly dictates otherwise.

As used herein, the term “comprising” is intended to mean that the compounds, compositions and processes include the recited elements, but not exclude others. “Consisting essentially of” when used to define compounds, compositions and processes, shall mean excluding other elements of any essential significance to the combination. Thus, a composition consisting essentially of the elements as defined herein would not exclude trace contaminants, e.g., from the isolation and purification method. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%, e.g., by using the prefix, “about.” It is to be understood, although not always explicitly stated that all numerical designations are preceded by the term “about.” It also is to be understood, although not always explicitly stated, that the reagents described herein are merely exemplary and that equivalents of such are known in the art.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂),-n-propyl-(CH₃CH₂CH₂—), isopropyl ((CH₃)₂CH),-n-butyl-(CH₃CH₂CH₂CH₂—), isobutyl ((CH₃)₂CHCH₂—), sec-butyl ((CH₃) (CH₃CH₂)CH),-t-butyl-((CH₃)₃C),-n-pentyl-(CH₃CH₂CH₂CH₂CH₂—), and neopentyl (CH₃)₃CH₂—).

### Ion Exchange and Esterification

In one aspect, provided herein is a process in which a diester of malonic acid is produced comprising:
a. fermentation to generate malonate as a salt;
b. separation of crude liquid malonate from cells;
c. optionally, ultrafiltration or nanofiltration to separate malonate from impurities with high molecular weight;
d. optionally, concentration by evaporation or reverse osmosis to concentrate the crude liquid malonate;
e. ion exchange with a solid ion exchange material to produce a stream of aqueous free malonic acid (that is separated from the original cation present in the malonic acid salt) and optionally further purified with respect to other broth impurities;
f. evaporation to minimize water content of the crude aqueous malonic acid;
g. mixing the resulting concentrate with alcohol and contacting it solid catalyst to generate a solution containing diester, monoester, and residual malonic acid;
h. distillation of the resulting solution to remove water as well as alcohol;
i. mixing the resulting material with alcohol and contacting it with solid catalyst to generate a solution containing diester, monoester, and residual malonic acid;
j. optionally, repeating steps h and l one or more times to maximize the amount of diester generated;
k. separating the diester product from other remaining components by fractional distillation;
l. optionally, recycling distilled alcohol after separating it from water by distillation, or swing adsorption of water onto an adsorbent, or pervaporation, or vapor permeation.

In one embodiment, the fermentation product contains malonate that is primarily a salt of sodium, calcium, or ammonium.

In another embodiment, the separation of crude liquid malonate from cells is accomplished by microfiltration, centrifugation, drum filtration, or belt filtration.

In another embodiment, steps b and c are combined, so that cells are separated from crude liquid malonate while processing the whole broth through an ultrafilter or nanofilter.

In another embodiment, a nanofilter is utilized in step c, described above, and the nanofilter material is selected so that it rejects >50% of the trehalose contained in the crude liquid malonate. In another embodiment, a nanofilter is utilized in step c, described above, and the nanofilter material is selected so that it rejects >10% or >30% of the glucose contained in the crude liquid malonate. In another embodiment, a nanofilter is utilized in step c and the nanofilter material is selected so that it rejects >10% or >30% of the succinate salts contained in the crude liquid malonate.

In another embodiment, ion exchange is accomplished by continuous ion exchange, using a valve and resin bed configuration designed to simulate a moving resin bed.

In another embodiment, ion exchange is performed with a cation exchange resin that is regenerated periodically with acid so that the malonate is protonated while contacting the resin.

In another embodiment, the acid used for regeneration is aqueous sulfuric acid.

In another embodiment, the fermentation product comprises malonate as an ammonium salt. In another embodiment, ammonium sulfate is generated as a co-product when the resin is periodically regenerated.

In another embodiment, ion exchange is performed with an anion exchange resin that is regenerated periodically with acid so that the malonate is protonated and eluted from the resin by the acid regenerant.

In another embodiment, optional step c is omitted and impurities of high molecular weight pass substantially through the anion exchange resin during malonate adsorption, separating them from malonate.

In another embodiment, step b as well as optional step c is omitted. In another embodiment, yeast and impurities of high molecular weight pass substantially through the anion exchange resin during malonate adsorption, separating them from malonate.

In another embodiment, the acid regenerant is aqueous sulfuric acid.

In another embodiment, the fermentation product comprises malonate as an ammonium salt. In another embodiment, ammonium sulfate is generated and eluted as a co-product while malonate is periodically adsorbed onto the resin.

In another embodiment, the ammonium sulfate eluted during malonate adsorption is a mixture of sulfuric acid, ammonium bisulfate, and ammonium sulfate containing between 0.7 and 2 mol ammonium per mol of sulfate.

In another embodiment, the alcohol used for the esterification is methanol, ethanol, propanol, isopropanol, butanol, pentanol, hexanol, phenol, or an alcohol containing less than 10 carbon atoms.

In another embodiment, the molar ratio of alcohol to malonate in the material contacted with catalyst in step g is at least 2, or at least 3, or at least 5, or at least 10.

In another embodiment, the solid catalyst is a cation resin that is primarily in the protonated form, which is prepared for use by contacting with acid.

In another embodiment, the diester product is stripped to remove low-boilers in a final distillation stage, to eliminate acetate esters or any other low-boilers generated by heat exposure during distillation.

In another embodiment, evaporation steps d and f are operated under sufficient vacuum to limit the boiling temperature to <50° C., or <75° C., or <100° C., to limit thermal decomposition of malonic acid or its salts during distillation.

In another embodiment, distillation steps h and k are operated under sufficient vacuum to limit the boiling temperature to <50° C., or <75° C., or <150° C., to limit thermal decomposition of malonic acid or its esters during distillation.

In another embodiment, the product is collected as a vapor side draw from the reboiler stage, or from a stage 1-5 stages above the reboiler stage.

In another embodiment, the final product is at least 95%, or at least 98%, or at least 99%, or at least 99.5% pure on a basis of weight percent purity. In another embodiment, the diester product contains <0.01 mg/kg of cyano-containing organic compounds, and/or <0.01 mg/kg of halogenated organic compounds. In another embodiment, the diester product contains >0.1 mg/kg of dialkyl succinate, and/or >0.1 mg/kg of dialkyl levulinate.

In another embodiment, the percent modern carbon of the 3 carbons of the resulting malonate diester originating from the malonate in the fermentation product is greater than 95%, or is essentially 100%, when measured using ¹⁴C radioisotope analysis corrected with standard methods such as delta ¹³C correction to correct for isotopic fractionation in the natural environment.

In another embodiment, provided herein is a process in which a diester of malonic acid is produced comprising the following elements:
ai. Fermentation to generate malonate as a salt;
bi. Separation of crude liquid malonate from cells;
ci. Optionally, ultrafiltration or nanofiltration to separate malonate from impurities with high molecular weight;
di. Crystallization of solid malonic acid from liquor;
ei. Filtration of solid malonic acid from liquor, and optionally drying of solid malonic acid crystals;
fi. Dissolution of the resulting crystals in alcohol;
gi. Contacting the resulting solution with solid catalyst to generate a solution containing diester, monoester, and residual malonic acid;
hi. Optionally, distillation of the resulting solution to remove water as well as alcohol;
ii. Optionally, mixing the resulting material with alcohol and contacting it with solid catalyst to generate a solution containing diester, monoester, and residual malonic acid;
ji. Optionally, repeating steps h and i one or more times to maximize the amount of diester generated;
ki. Separating the diester product from other remaining components by fractional distillation;
li. Recycling distilled alcohol after separating it from water by distillation, or swing adsorption of water onto an adsorbent, or pervaporation, or vapor permeation.

Other methods for continuous ion exchange of other diacids are reported in US 9,233,906 to Gerberding, et al., entitled “PURIFICATION OF SUCCINIC ACID FROM THE FERMENTATION BROTH CONTAINING AMMONIUM SUCCINATE”, the disclosure of which is hereby incorporated by reference herein in its entirety, which methods can be adapted by a person skilled in the art based on the teaching provided herein.

### EXAMPLES

The invention is illustrated and not limited by these examples.

### Example 1. Ion Exchange Chromatography with Anion Exchange Resin

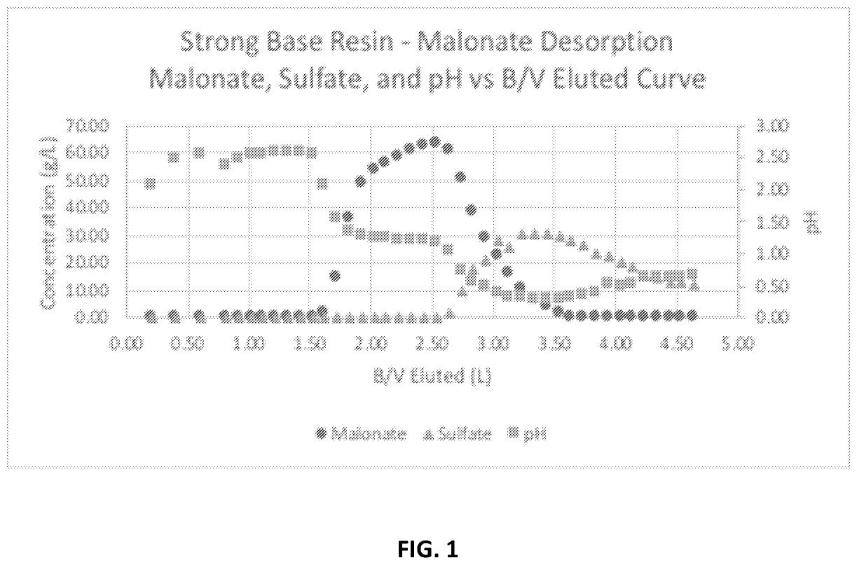

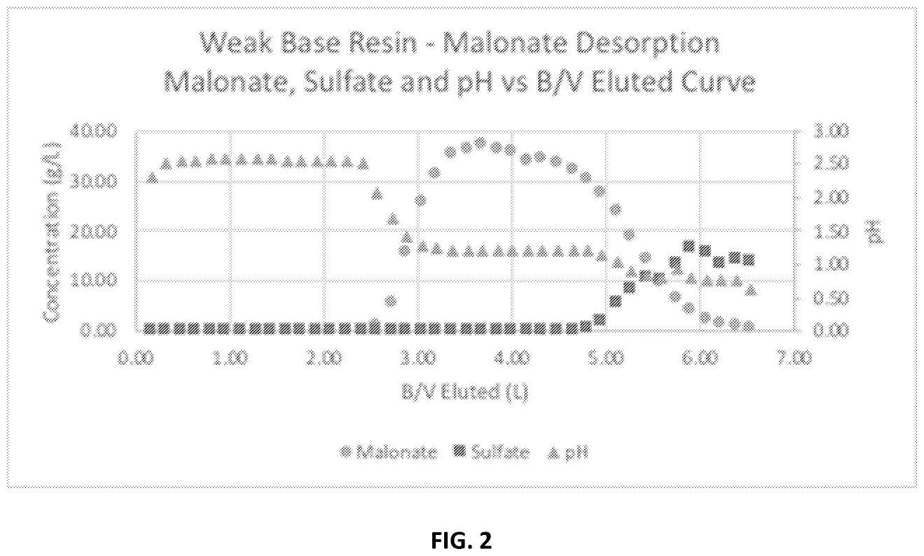

Three types of clarified fermentation broths (centrifuged, ultrafiltered (UF), and nanofiltered (NF)) were used as feeds for the anion exchange study. The concentration of malonic acid in the feed ranged from 40 g/L to 160 g/L. The anion exchange resin was initially in sulfate form. The feed rate to the column ranged from 1 to 4 bed volumes per hour (BV/h). The ion-exchange process was performed at 25° C. and 1 atm. The resin bed volume was around 3-4 L and 0.5 L of a sample was taken every 3 minutes to monitor flow rate, pH, density, and concentrations of malonate and sulfate. After the feed was exhausted, the bed of resin was rinsed with 5-10 bed volumes of water to elute the excess malonate and sulfate in the column until the eluate pH stabilized. After the water rinse, a malonic acid solution was eluted from the resin bed by feeding 2.5 wt % to 10 wt % sulfuric acid. FIGS. 1 and 2 illustrate desorption curves of malonic acid from strong base and weak base anion exchange resin, respectively.

### Example 2. Ion Exchange Chromatography with Cation Exchange Resin

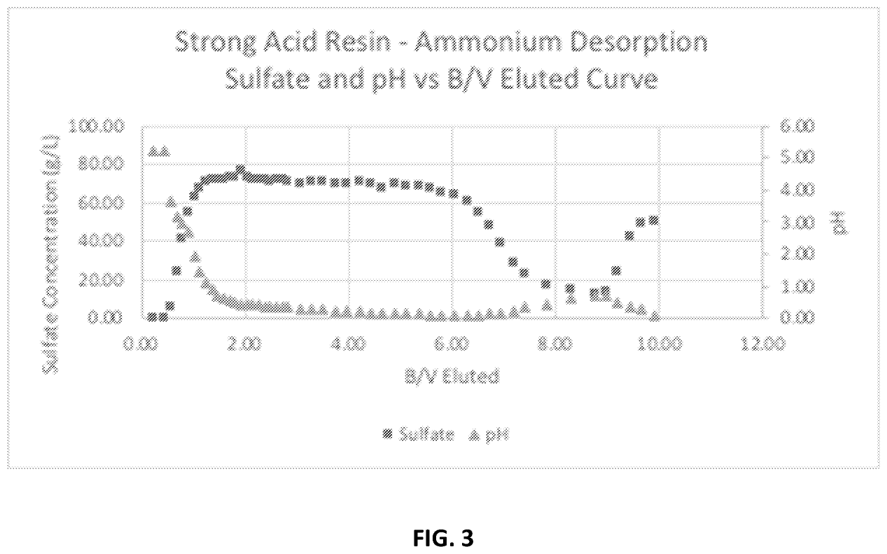

The same three types of broth used in Example 1 were also used as feeds to a cation exchange resin to bind ammonium cation and yield malonic acid solution in the effluent. The concentration of malonic acid in the feed ranged from 40 g/L to 160 g/L. The cation exchange resin was in hydrogen form. The feed rate to the column ranged from 1 to 4 BV/h. The ion-exchange process was performed at 25° C. and 1 atm. The resin bed volume was approximately 3-4 L, and 0.5 L samples were taken every 3 minutes to monitor feed rate, pH, density, and concentrations of ammonium and malonate. After the feed was exhausted, the bed of resin was rinsed with 5-10 bed volumes of water to wash out the malonate and other impurities. After the water rinse, the resin bed was washed with 2.5 wt % to 10 wt % sulfuric acid to elute ammonium sulfate. FIG. 3 shows an example of a desorption curve of ammonium from a strong acid exchange resin.

### Example 3. Primary Fixed-Bed Reaction

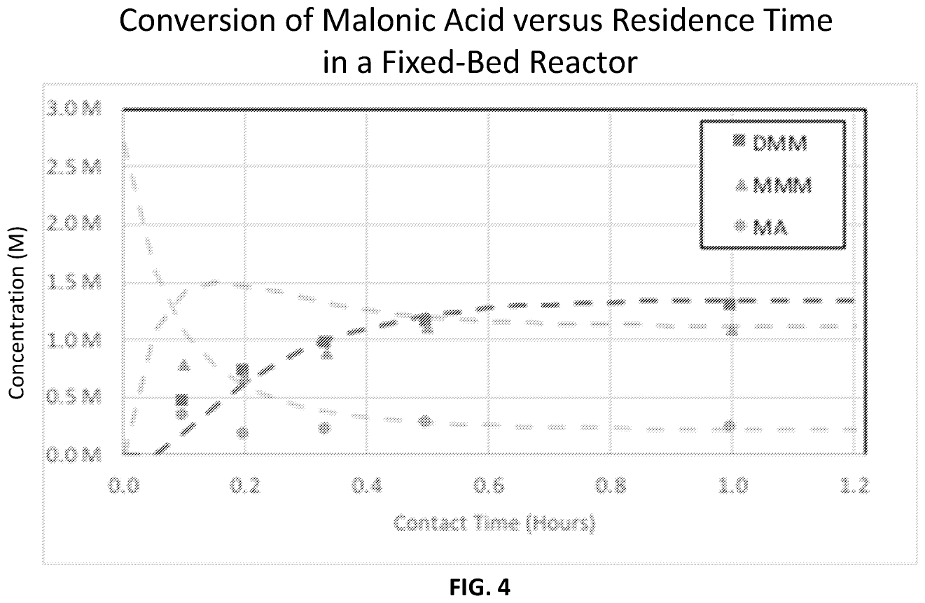

1465 grams of methanol (45.8 mol) was mixed with 1066 grams of a 45 weight-% fermentation-derived solution of malonic acid (4.58 moles). The solution was pumped through a fixed-bed reactor with the specifications shown in Table 1 using a piston pump. The backpressure of the system was controlled at 40 psi using a backpressure regulator to ensure all methanol remains in the liquid phase. A pressure drop of 5 psi was measured across the reactor at the specified flow rate. Referring briefly to FIG. 4, the flow rate was chosen such that the reaction equilibrium would be achieved within the residence time of the reactor. The output composition, conversion of malonic acid, and yield of dimethyl malonate is summarized in Table 2.

TABLE 1
Fixed-bed reactor specifications and operating parameters.

| Specification | Value | |
|---|---|---|
| Reactor | 1″ | |
| Diameter | | |
| Reactor Height | 36″ | |
| Bed Height | 9.3″ | |
| Bed Volume | 120 | mL |
| Catalyst | Purolite CT151 | |
| Temperature | 80 | C. |
| Flow rate | 2 | mL/min |

TABLE 2
Reactor product composition, conversion and yield
of a fermentation-derived malonic acid solution.

| Component | Composition |
|---|---|
| Malonic Acid | 0.9 wt-% |
| Mono-methyl Malonate | 7.6 wt-% |
| Dimethyl Malonate | 13.1 wt-% |
| Water | 29.3 wt-% |
| Methanol | 40.4 wt-% |
| Conversion | 94.8% |
| Yield | 57.6% |

### Example 4. Methanol and Water Distillation

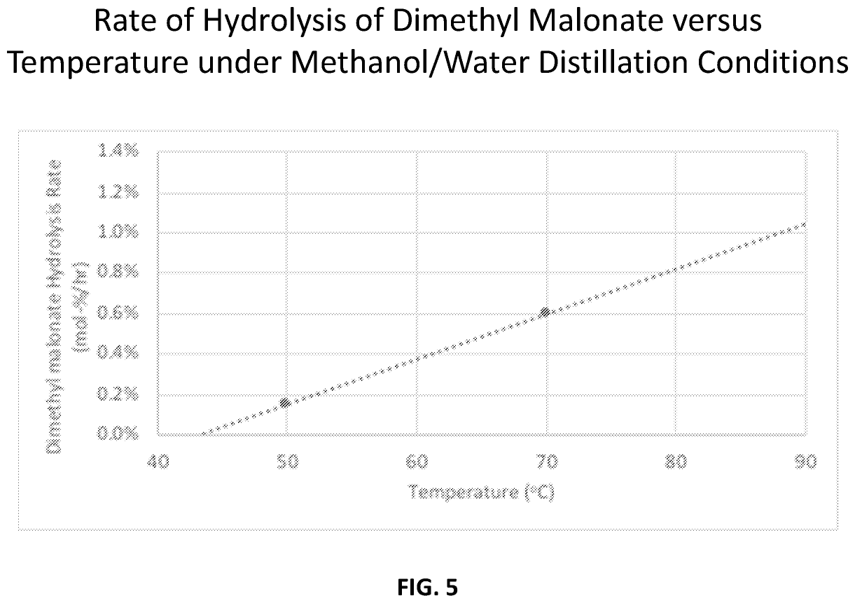

Methanol and water were removed from the reactor product summarized in Example 3 to achieve a final water content between 4-7 weight % measured by Karl Fischer titration. The distillation was performed in the same apparatus as the final product distillation. The pressure of the distillation was reduced to ensure a pot temperature below 50° C. was maintained throughout the distillation. The rate of hydrolysis of DMM versus temperature for the compositions during distillation are shown in FIG. 5 below. Once the final water specification was achieved, the temperature was reduced and pressure increased to atmospheric for further processing. The final composition of the distillation pot is summarized in Table 3.

TABLE 3
Composition of a fixed-bed reactor product that has been
distilled to a final water content of 5.9 weight-%.

| Component | Composition | |
|---|---|---|
| Malonic Acid | 3.1 | wt-% |
| Mono-methyl malonate | 34.5 | wt-% |
| Dimethyl malonate | 56.5 | wt-% |
| Water | 5.9 | wt-% |
| Methanol | 0.0 | wt-% |

### Example 5. Polishing Fixed-bed Reaction

A second fixed-bed reaction was carried out on the methanol and water removed distillation product to achieve a dimethyl malonate yield of greater than 95%. The specifications and operating parameters of this fixed-bed reaction are identical to the primary fixed-bed reaction. An additional 10 molar equivalents of methanol to malonate equivalents is added to the water removed product. The solution was fed through the reactor at 2 mL/min at a temperature of 80° C. and collected for further processing. The final weight composition is summarized in Table-4.

TABLE 4
Polishing fixed-bed reactor product weight
composition, conversion, and yield.

| Component | Composition |
|---|---|
| Malonic Acid | 0.0 wt-% |
| Mono-methyl Malonate | 1.0 wt-% |
| Dimethyl Malonate | 27.9 wt-% |
| Water | 3.16 wt-% |
| Methanol | 67.9 wt-% |
| Conversion | 99.9% |
| Yield | 96.0% |

### Example 6. Final Product Distillation

The final batch fractional distillation was performed on 3.87 kg (4.66 L) of fermentation-derived material with the intention of reaching a dimethyl malonate (DMM) purity specification of 99.6-99.8 weight-%. Three successive separations were targeted; removal of methanol, removal of water, and then isolation of DMM from heavy impurities. The operating parameters, feed composition, and sample collection information are tabulated below.

TABLE 5
Operating Parameters.

| No. of theoretical stages | 22 | |
|---|---|---|
| Column packing | Sulzer DX | |
| Operating pressure | 20-150 | torr |
| Operating temperature | 32-150° | C. |

| | |
|---|---|
| Reflux ratio | 0.5-10 |

Product fractions were collected, and their purities were measured via gas chromatography, as tabulated below.

TABLE 6
Final Product Composition.

| Peak | | Retention | | Relative |
|---|---|---|---|---|
| Number | Peak Name | Time | GC Area | Area |
| 1 | Methanol | 2.22 | 0.165 | 0.06% |
| 2 | Methyl Acetate | 3.762 | 0.075 | 0.03% |
| 3 | Acetic Acid | 4.303 | 0.141 | 0.05% |
| 4 | Methyl Lactate | 6.25 | 0.034 | 0.01% |
| 5 | Methyl Hydroxybutyrate | 7.337 | 0.024 | 0.01% |
| 6 | 2-cyclopentene-1,4-dione | 7.527 | 0.019 | 0.01% |
| 7 | Dimethyl Malonate | 7.813 | 267.658 | 99.80% |
| 8 | Dimethyl Methylmalonate | 8.145 | 0.045 | 0.02% |
| 9 | Dimethyl Fumarate | 8.348 | 0.034 | 0.01% |

1. A process of ion exchange comprising:
contacting malonic acid or a salt thereof and a cation or anion exchange resin
wherein the cation exchange resin is regenerated periodically with an acid into the protonated form so that the malonic acid salt is protonated while contacting the resin and reports to the raffinate stream as an aqueous malonic acid solution free of a cation and the cation is adsorbed onto the resin to be eluted as the corresponding salt of the regenerating acid, or
wherein the anion exchange resin is regenerated periodically with an acid so that the malonic acid that has previously been adsorbed on the resin is eluted from the resin and the conjugate base of the regenerating acid is adsorbed on the resin ready for the next cycle
wherein the ion exchange is accomplished by continuous ion exchange, using a resin bed system configuration designed to simulate a resin bed moving countercurrent-wise to the fluid flow.

2. The process according to claim 1, wherein the malonic acid salt comprises a sodium, calcium, or ammonium salt.

3. A process according to claim 1, in which the malonic acid or salt thereof is included in a crude aqueous malonate fermentation product.

4. The process according to claim 1, in which the malonic acid or salt thereof is included in a crude aqueous malonate fermentation product, which is separated from fermenting cells by microfiltration, centrifugation, drum filtration, or belt filtration.

5. The process according to claim 1, in which the malonic acid or salt thereof is included in a crude aqueous malonate fermentation product, which is separated from fermenting by filtering a fermentation broth through an ultrafilter or nanofilter.

6. A process according to claim 5, in which a nanofilter is utilized and the nanofilter material is selected so that it rejects >50% of trehalose contained in the fermentation broth.

7. A process according to claim 5, in which a nanofilter is utilized and the nanofilter material is selected so that it rejects >10% or >30% of the glucose contained in the fermentation broth.

8. A process according to claim 5, in which a nanofilter is utilized and the nanofilter material is selected so that it rejects >10% or >30% of the succinate salts contained in the fermentation broth.

9. A process according to claim 1, in which ion exchange is performed with a cation exchange resin that is regenerated periodically with acid so that the malonate is protonated while contacting the resin.

10. A process according to claim 10, in which the acid used for regeneration is aqueous sulfuric acid.

11. A process according to claim 10, in which the fermentation product contains malonate primarily as an ammonium salt, so that ammonium sulfate is generated as a co-product when the resin is periodically regenerated.

12. A process according to claim 1, in which ion exchange is performed with an anion exchange resin that is regenerated periodically with acid so that the malonate is protonated and eluted from the resin during the acid regeneration.

13. A process according to claim 12, in which impurities of high molecular weight pass substantially through the anion exchange resin during malonate adsorption on the anion exchange resin, thereby separating the impurities from malonate.

14. A process according to claim 12, in which a fermenting cell such as yeast and impurities of high molecular weight pass substantially through the anion exchange resin during malonate adsorption on the anion exchange resin, thereby separating the fermenting cells and impurities from the malonate.

15. A process according to claim 12, in which the acid regenerant is aqueous sulfuric acid.

16. A process according to claim 15, in which the malonate salt comprises an ammonium salt, so that ammonium sulfate is generated and eluted as a co-product while malonate is adsorbed onto the resin.

17. A process according to claim 1, wherein the separated malonic acid is esterified with an alcohol under conditions suitable to form a malonate ester.

18. The process according to claim 17, in which the alcohol used for the esterification is methanol, ethanol, propanol, isopropanol, butanol, pentanol, hexanol, phenol, or an alcohol containing less than 10 carbon atoms.

19. A process according to claim 17, in which the molar ratio of alcohol to malonate in the material contacted with catalyst in step g is at least 2, or at least 3, or at least 5, or at least 10.

20. A process according to claim 17, in which the esterification employs a solid catalyst, such as a cation resin that is primarily in the protonated form, which is prepared for use by contacting with acid.

21. A process according to claim 17, in which the malonate ester such as a diester product is stripped to remove low-boilers in a final distillation stage, to eliminate acetate esters or any other low-boilers generated by heat exposure during distillation.

22. A process according to claim 17, in which the final malonate ester product is at least 95%, or at least 98%, or at least 99%, or at least 99.5% pure on a basis of weight percent purity.

23. A process according to claim 17, in which the final malonate ester such as a diester product contains <0.01 mg/kg of cyano-containing organic compounds, and/or <0.01 mg/kg of halogenated organic compounds.

24. A process according to claim 17, in which the final malonate ester such as a diester product contains >0.1 mg/kg of dialkyl succinate, and/or >0.1 mg/kg of dialkyl levulinate.

25. A process according to claim 17, in which the percent modern carbon of the 3 carbons of the resulting malonate ester such as a diester originating from the malonate in the fermentation product is greater than 95%, or is essentially 100%, when measured using ¹⁴C radioisotope analysis corrected with standard methods such as delta ¹³C correction to correct for isotopic fractionation in the natural environment.

26. A process in which a diester of malonic acid is produced comprising the following elements:
a. fermentation to generate malonate as a salt;
b. separation of crude liquid malonate from cells;
c. optionally, ultrafiltration or nanofiltration to separate malonate from impurities with high molecular weight;
d. crystallization of solid malonic acid from liquor;
e. filtration of solid malonic acid from liquor, and optionally drying of solid malonic acid crystals;
f. dissolution of the resulting crystals in alcohol;
g. contacting the resulting solution with solid catalyst to generate a solution containing diester, monoester, and residual malonic acid;
h. optionally, distillation of the resulting solution to remove water as well as alcohol;
i. optionally, mixing the resulting material with alcohol and contacting it with solid catalyst to generate a solution containing diester, monoester, and residual malonic acid;
j. optionally, repeating steps h and i one or more times to maximize the amount of diester generated;
k. separating the diester product from other remaining components by fractional distillation;
l. recycling distilled alcohol after separating it from water by distillation, or swing adsorption of water onto an adsorbent, or pervaporation, or vapor permeation.
